(19) United States
(12) Patent Application Publication
(10) Pub. No.: US 2012/0184624 A1
(43) Pub. Date: Jul. 19, 2012

(54) Treatment of Alzheimer's Disease

(76) Inventors: Susann Schweiger, (US); Rainer Schneider, (US); Hartmut Glossmann, (US)

(21) Appl. No.: 13/328,108

(22) Filed: Dec. 16, 2011

Related U.S. Application Data
(60) Provisional application No. 61/424,196, filed on Dec. 17, 2010.

(51) Int. Cl. A61K 31/155; A61P 25/28

(52) U.S. Cl. 514/635

(57) ABSTRACT

The present invention relates to a method of treating and/or preventing Alzheimer's disease and/or other tauopathies.

### RELATED APPLICATION

This application claims the benefit of U.S. Provisional Patent Application Ser. No. 61/424,196, filed Dec. 17, 2010, the disclosure of which is incorporated by reference herein in its entirety.

### FIELD OF INVENTION

The present invention relates to a method of treating and/or preventing Alzheimer's disease and/or other tauopathies.

### BACKGROUND TO THE INVENTION

Alzheimer's disease (AD) is the most common cause of dementia in the elderly an is characterised by a decline in cognitive function, that progresses slowly and results in symptoms such as memory loss and disorientation. Death occurs, on average, 9 years after diagnosis. The incidence of AD increases with age, so that while about 5% of people over the age of 70 are sufferers, this FIGURE increases to 20% of those over 80 years old.

Existing treatments exclusively target the primary symptoms of AD. Diseased neurons may release insufficient or excessive amounts of particular neurotransmitters, and so current drugs are aimed at increasing neurotransmitter levels or at reducing the stimulation of nerve cells by neurotransmitters. Although these drugs provide some improvement in the symptoms of AD, they fail to address the underlying cause of the disease.

Formation of paired helical filaments (PHFs) in the brain is a characteristic hallmark in the pathogenesis of Alzheimer's Disease (AD) and related tauopathies. The major protein component of PHFs is the hyperphosphorylated form of tau, which in its normo-phosphorylated form is a microtubule-associated protein, that stimulates and stabilizes microtubule assembly [reviewed in (1)]. While tau normally contains 2-3 moles of phosphates per mole, tau phosphorylation levels in AD brains are 3- to 4-fold higher. Upon hyperphosphorylation, tau dissociates from the microtubules and subsequently sequesters normal tau and other microtubule-associated proteins, which inhibits assembly and depolymerizes microtubules (2).

Tau in PHFs has been found to be differently phosphorylated at more than 30 serine/threonine residues compared to normal tau (3). Among the classical phosphoseryl/phosphothreonyl phosphatases, PP2A seems to be the major tau phosphatase in the brain (4-8). Indeed reduction of both—activity and expression of PP2A—has been observed in AD brains repeatedly (9-13). This makes PP2A activity a valuable target for the development of a potential therapy for AD.

### SUMMARY OF THE INVENTION

The present invention is based in part on initial observations that compounds that reduce the phosphorylation of PP2A dependent tau epitopes may find application in preventing and/or treating tau related disorders—tauopathies, such as Alzheimer's disease. Indeed the present inventor has identified compounds which interfere with the association of the catalytic subunit of PP2A (PP2Ac) to the so-called MID1-α4 protein complex, which regulates degradation of PP2Ac and thereby influences PC2A activity.

Thus, in a first aspect there is provided a method of preventing and/or treating a tau related disorder (i.e. a tauopathy) comprising the step of administering to a subject a compound which is capable inhibiting assembly of a MID1-α4-PP2Ac complex.

Based on the inventors observations compounds which serve to inhibit the assembly of the MID1-α4-PP2Ac complex, as a consequence of present proteosome degradation of said complex and hence activate/prolong PP2-activity and hence dephosphorylation of tau.

Diseases which may be prevented and/or treated by the compounds of the present invention include Progressive supranuclear palsy, Dementia pugilistica (chronic traumatic encephalopathy), Frontotemporal dementia and parkinsonism linked to chromosome 17 however without detectable β-amyloid plaques, Lytico-Bodig disease (Parkinson-dementia complex of Guam), Tangle-predominant dementia, with NFTs similar to AD, but without plaques, Ganglioglioma and gangliocytoma, Meningioangiomatosis, Subacute sclerosing panencephalitis, as well as lead encephalopathy, tuberous sclerosis, Hallervorden-Spatz disease, and lipofuscinosis. However, the most prevalent disease associated with tau aggregation is Alzheimer's disease and hence this is the preferred disease to be prevented and/or treated.

The compounds which may find application in accordance with the present invention include one or more compounds or classes selected from the group consisting of: Biguanides, such as Metformin, Phenformin, Buformin and Proguanil; Guanidines, such as Galegin and Hydroxygalegin; Berberine; Alkylphosphocholin (Erucylphosphohomocholine); Spermidine; Catechines; Quercetin; Naringin; Apigenin; Luteolin; Procyanidin; Isoflavones, including Genistein and Daizein; Resveratrol; (Dihydroxyphenyl)ethanol; Curcumin; Alpha-Tocopherol; Forskolin; Inositol, Li+; Troglitazone; Valproate; Ceramide; Alkylphospholipide; Palmitate; Linoleic acid; Phosphatidic acid; Lysophosphatidic acid; FTY720; Endostatin; Vasoinhibin; Somatostatin; Adiponectin; SAM; methionine; THF; Natriumselenate; Sulforaphane; Salicylic acid and Apomorphine.

In a preferred embodiment, the compound or compounds for use in the present invention are a biguanidine compound or derivative, including Metformin, Phenformin, Buformin and Proguanil, especially Metformin and Phenformin.

The compounds of the present invention may be administered above or in combination with other therapeutic agents. Other therapeutic agents (e.g. agents designed to target amyloid placques for example) may be administered together (whether concurrently or at different time intervals) with the compounds/compositions of the invention. For the case of active compounds combined with other therapies the two or more treatments may be given in individually varying dose schedules and via different routes.

The combination of such agents with a compound of the present invention would be at the discretion of the physician who would select dosages using his common general knowledge and dosing regimens known to a skilled practitioner.

Where a compound of the invention is administered in combination therapy with one, two, three, four or more, preferably one or two, preferably one other therapeutic agents, the compounds can be administered simultaneously or sequentially. When administered sequentially, they can be administered at closely spaced intervals (for example over a period of 5-10 minutes) or at longer intervals (for example 1, 2, 3, 4 or more hours apart, or even longer period apart where required), the precise dosage regimen being commensurate with the properties of the therapeutic agent(s).

The patient is typically an animal, e.g. a mammal, especially a human.

It is to be understood that the compounds of the present invention are to be administered in a therapeutically or prophylactically effective amount. By a therapeutically or prophylactically effective amount is meant one capable of achieving the desired response, and will be adjudged, typically, by a medical practitioner. The amount required will depend upon one or more of at least the active compound(s) concerned, the patient, the condition it is desired to treat or prevent and the formulation of order of from 1 μg to 1 g of compound per kg of body weight of the patient being treated.

Different dosing regiments may likewise be administered, again typically at the discretion of the medical practitioner. The low toxicity of many of the compounds of the invention, allow for at least daily administration although regimes where the compound(s) is (or are) administered more infrequently, e.g. every other day, weekly or fortnightly, for example, are also embraced by the present invention.

By treatment is meant herein at least an amelioration of a condition suffered by a patient; the treatment need not be curative (i.e. resulting in obviation of the condition). Analogously references herein to prevention or prophylaxis herein do not indicate or require complete prevention of a condition; its manifestation may instead be reduced or delayed via prophylaxis or prevention according to the present invention.

For use according to the present invention, the compounds or pharmaceutically acceptable salt, solvate, ester, or amide thereof described herein may be presented as a pharmaceutical formulation, comprising the compound, or pharmaceutically acceptable salt, solvate, ester, or amide thereof, together with one or more pharmaceutically acceptable carriers therefor and optionally other therapeutic and/or prophylactic ingredients. Any carrier(s) are acceptable in the sense of being compatible with the other ingredients of the formulation and not deleterious to the recipient thereof.

Examples of pharmaceutically acceptable salts of the compounds according to the invention include acid addition salts formed with organic carboxylic acids such as acetic, lactic, tartaric, maleic, citric, pyruvic, oxalic, fumaric, oxaloacetic, isethionic, lactobionic and succinic acids; organic sulfonic acids such as methanesulfonic, ethanesulfonic, benzenesulfonic and p-toluenesulfonic acids and inorganic acids such as hydrochloric, sulfuric, phosphoric and sulfamic acids.

Pharmaceutically acceptable salt, solvate, ester, or amide thereof may be physiologically functional derivatives of the compounds of the present invention. Such physiologically functional derivatives may also be referred to as “pro-drugs” or “bioprecursors”. Physiologically functional derivatives of compounds of the present invention include in vivo hydrolysable esters or amides, particularly esters. Determination of suitable pharmaceutically acceptable esters and amides is well within the ability of those skilled in the art.

It may be convenient or desirable to prepare, purify, and/or handle a corresponding solvate of the compounds described herein, which may be used in the any one of the uses/methods described. The term solvate is used herein to refer to a complex of solute, such as a compound or salt of the compound, and a solvent. If the solvent is water, the solvate may be termed a hydrate, for example a mono-hydrate, di-hydrate, tri-hydrate etc, depending on the number of water molecules present per molecule of substrate.

It will be appreciated that the compounds of the present invention may exist in various stereoisomeric forms and the compounds of the present invention as hereinbefore defined include all stereoisomeric forms and mixtures thereof, including enantiomers and racemic mixtures. The present invention includes within its scope the use of any such stereoisomeric form or mixture of stereoisomers, including the individual enantiomers of the compounds of formulae (I) or (II) as well as wholly or partially racemic mixtures of such enantiomers.

The compounds of the present invention may be prepared using reagents and techniques readily available in the art and/or exemplary methods as described hereinafter.

Pharmaceutical formulations include those suitable for oral, topical (including dermal, buccal and sublingual), rectal or parenteral (including subcutaneous, intradermal, intramuscular, intraperitoneal, and intravenous, subcutaneous, epidural, topical, transdermal, parenteral, intrathecal, vaginal, rectal, colorectal, oral, intracranial, retroorbital, intrasternal), nasal and pulmonary administration e.g., by inhalation. The formulation may, where appropriate, be conveniently presented in discrete dosage units and may be prepared by any of the methods well known in the art of pharmacy. Methods typically include the step of bringing into association an active compound with liquid carriers or finely divided solid carriers or both and then, if necessary, shaping the product into the desired formulation.

Pharmaceutical formulations suitable for oral administration wherein the carrier is a solid are most preferably presented as unit dose formulations such as boluses, capsules or tablets each containing a predetermined amount of active compound. A tablet may be made by compression or moulding, optionally with one or more accessory ingredients. Compressed tablets may be prepared by compressing in a suitable machine an active compound in a free-flowing form such as a powder or granules optionally mixed with a binder, lubricant, inert diluent, lubricating agent, surface-active agent or dispersing agent. Moulded tablets may be made by moulding an active compound with an inert liquid diluent. Tablets may be optionally coated and, if uncoated, may optionally be scored. Capsules may be prepared by filling an active compound, either alone or in admixture with one or more accessory ingredients, into the capsule shells and then sealing them in the usual manner. Cachets are analogous to capsules wherein an active compound together with any accessory ingredient(s) is sealed in a rice paper envelope. An active compound may also be formulated as dispersible granules, which may for example be suspended in water before administration, or sprinkled on food. The granules may be packaged, e.g., in a sachet. Formulations suitable for oral administration wherein the carrier is a liquid may be presented as a solution or a suspension in an aqueous or non-aqueous liquid, or as an oil-in-water liquid emulsion.

Formulations for oral administration include controlled release dosage forms, e.g., tablets wherein an active compound is formulated in an appropriate release-controlling matrix, or is coated with a suitable release-controlling film. Such formulations may be particularly convenient for prophylactic use.

Pharmaceutical formulations suitable for rectal administration wherein the carrier is a solid are most preferably presented as unit dose suppositories. Suitable carriers include cocoa butter and other materials commonly used in the art. The suppositories may be conveniently formed by admixture of an active compound with the softened or melted carrier(s) followed by chilling and shaping in moulds.

Pharmaceutical formulations suitable for parenteral administration include sterile solutions or suspensions of an active compound in aqueous or oleaginous vehicles.

Injectible preparations may be adapted for bolus injection or continuous infusion. Such preparations are conveniently presented in unit dose or multi-dose containers which are sealed after introduction of the formulation until required for use. Alternatively, an active compound may be in powder form which is constituted with a suitable vehicle, such as sterile, pyrogen-free water, before use.

An active compound may also be formulated as long-acting depot preparations, which may be administered by intramuscular injection or by implantation, e.g., subcutaneously or intramuscularly. Depot preparations may include, for example, suitable polymeric or hydrophobic materials, or ion-exchange resins. Such long-acting formulations are particularly convenient for prophylactic use.

Formulations suitable for pulmonary administration via the buccal cavity are presented such that particles containing an active compound and desirably having a diameter in the range of 0.5 to 7 microns are delivered in the bronchial tree of the recipient.

As one possibility such formulations are in the form of finely comminuted powders which may conveniently be presented either in a pierceable capsule, suitably of, for example, gelatin, for use in an inhalation device, or alternatively as a self-propelling formulation comprising an active compound, a suitable liquid or gaseous propellant and optionally other ingredients such as a surfactant and/or a solid diluent. Suitable liquid propellants include propane and the chlorofluorocarbons, and suitable gaseous propellants include carbon dioxide. Self-propelling formulations may also be employed wherein an active compound is dispensed in the form of droplets of solution or suspension.

Such self-propelling formulations are analogous to those known in the art and may be prepared by established procedures. Suitably they are presented in a container provided with either a manually-operable or automatically functioning valve having the desired spray characteristics; advantageously the valve is of a metered type delivering a fixed volume, for example, 25 to 100 microlitres, upon each operation thereof.

As a further possibility an active compound may be in the form of a solution or suspension for use in an atomizer or nebuliser whereby an accelerated airstream or ultrasonic agitation is employed to produce a fine droplet mist for inhalation.

Formulations suitable for nasal administration include preparations generally similar to those described above for pulmonary administration. When dispensed such formulations should desirably have a particle diameter in the range 10 to 200 microns to enable retention in the nasal cavity; this may be achieved by, as appropriate, use of a powder of a suitable particle size or choice of an appropriate valve. Other suitable formulations include coarse powders having a particle diameter in the range 20 to 500 microns, for administration by rapid inhalation through the nasal passage from a container held close up to the nose, and nasal drops comprising 0.2 to 5% w/v of an active compound in aqueous or oily solution or suspension.

It should be understood that in addition to the aforementioned carrier ingredients the pharmaceutical formulations described above may include, an appropriate one or more additional carrier ingredients such as diluents, buffers, flavouring agents, binders, surface active agents, thickeners, lubricants, preservatives (including anti-oxidants) and the like, and substances included for the purpose of rendering the formulation isotonic with the blood of the intended recipient.

Pharmaceutically acceptable carriers are well known to those skilled in the art and include, but are not limited to, 0.1 M and preferably 0.05 M phosphate buffer or 0.8% saline. Additionally, pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Preservatives and other additives may also be present, such as, for example, antimicrobials, antioxidants, chelating agents, inert gases and the like.

Therapeutic formulations for veterinary use may conveniently be in either powder or liquid concentrate form. In accordance with standard veterinary formulation practice, conventional water soluble excipients, such as lactose or sucrose, may be incorporated in the powders to improve their physical properties. Thus particularly suitable powders of this invention comprise 50 to 100% w/w and preferably 60 to 80% w/w of the active ingredient(s) and 0 to 50% w/w and preferably 20 to 40% w/w of conventional veterinary excipients. These powders may either be added to animal feedstuffs, for example by way of an intermediate premix, or diluted in animal drinking water.

Liquid concentrates of this invention suitably contain the compound or a derivative or salt thereof and may optionally include a veterinarily acceptable water-miscible solvent, for example polyethylene glycol, propylene glycol, glycerol, glycerol formal or such a solvent mixed with up to 30% v/v of ethanol. The liquid concentrates may be administered to the drinking water of animals.

The contents of each literature and patent reference referred to herein is hereby incorporated by reference in its entirety as if the entire contents of each of these references were recited herein in full.

### BRIEF DESCRIPTION OF THE DRAWINGS

FIG. 1 shows that Metformin decreases the phosphorylation of S6 ribosomal protein. (A) Primary cortical neurons of wildtype mice were treated with metformin (Met) at the indicated concentrations for 16 hours, (B) Primary cortical neurons of wildtype mice were treated with or without 2.5 mM metformin (Met) for 1 and 4 hours, (C) H4 cells were treated with or without 2.5 mM metformin (Met) for 16 hours. Cell lysates were analysed by Western blotting using an antibody to phosphorylated-S6 (241/244). Band intensities were normalized to either actin or total tau (Tau5). In each case a representative blot is shown along with the quantification of three independent experiments. n=3, * p<0.01.

FIG. 2 shows that Metformin induces de-phosphorylation of tau at PP2A sensitive sites. Representative Western blots and quantifications of three or four independent experiments are shown in A-D, F and G. Band intensities were normalized to actin and compared to total tau (Tau 5). (A) Primary cortical neurons of wildtype mice were treated with 2.5 mM metformin (Met) over increasing time intervals. Cell lysates were analysed by Western blot using antibodies detecting phosphorylation at specific, PP2A sensitive tau sites (Ser 202, Ser 262, Ser 356). n=3. (B) Primary cortical neurons from wildtype mice were treated with increasing concentration of metformin (Met) over 4 hours. Cell lysates were analysed by Western blot using an antibody detecting phosphorylation at Ser 202. n=4. (C) Primary cortical neurons of wildtype mice were treated with 2.5 mM metformin (Met) over increasing time intervals. Cell lysates were analysed by Western blot using an antibody detecting phosphorylation at the PP2A insensitive tau site Ser 396. n=3. (D) Primary cortical neurons of transgenic mice expressing human tau instead of murine tau were treated with increasing concentration of Metformin over 4 hours. Cell lysates were analysed by Western blot using an antibody detecting phosphorylation at Ser 202. n=3. (E) TUNEL assay of primary cortical neurons of wildtype mice treated with increasing concentrations of metformin. Percentages of apoptotic cells are shown. (F) Primary cortical neurons of wildtype mice were treated with low concentrations of metformin (Met) over 24 hours. Cell lysates were analysed by Western blot using an antibody detecting phosphorylation at Ser 202. n=3. (G) Primary cortical neurons of wildtype mice were treated with 2.5 mM Metformin (Met) over increasing time intervals in medium without insulin. Cell lysates were analysed by Western blot using an antibody detecting phosphorylation at Ser 202. n=3, * p<0.05.

FIG. 3 shows that Inhibition of PP2A blocks the Metformin effect on the phosphorylation of tau. Representative Western blots and quantifications of three or four independent experiments are shown in the subsequent experiments. Band intensities were normalized to actin and compared to total tau (Tau 5). (A) Primary cortical neurons of wildtype mice were treated with either okadaic acid (OA)(10 nM), only metformin (Met) (2.5 mM) or with okadaic acid (10 nM) prior to metformin (2.5 mM) for 4 hours. Cell lysates were analysed by Western blot using an antibody detecting phosphorylation at a specific, PP2A sensitive tau site. n=4, * p<0.05. (B) Primary cortical neurons of wildtype mice were treated with either fostriecin (Fost)(1 μM), only metformin (Met) (2.5 mM) or with fostriecin (1 μM) prior to metformin (2.5 mM) over 4 hours. Cell lysates were analysed by Western blot using an antibody detecting phosphorylation at a specific, PP2A sensitive tau site, n=3, * p<0.05.

FIG. 4 shows that Metformin effects on the phosphorylation of tau are AMPK independent. Representative Western blots and quantifications of 3-5 independent experiments are shown in the subsequent experiments. (A) Primary cortical neurons of wildtype mice were treated with either 2.5 mM of metformin (Met) or with 5 mM of AICAR over increasing time intervals. Cell lysates were analysed by Western blot using an antibody detecting phosphorylation of the AMPK target ACC. Band intensities were compared to total ACC. n=4. (B) Primary cortical neurons of wildtype mice were treated with 2.5 mM metformin (Met) over increasing time intervals. Cell lysates were analysed by Western blot using an antibody detecting phosphorylated AMPK (pThr 172). Band intensities were compared to total AMPK. n=3. (C) Primary cortical neurons of wildtype mice were treated with increasing concentrations of Metformin (Met) over 24 hours. Cell lysates were analysed by Western blot using an antibody detecting phosphorylation of AMPK (pThr 172). Band intensities were normalized to AMPK total. n=3. (D) Primary cortical neurons of wildtype mice were treated with 5 mM of AICAR over increasing time intervals. Cell lysates were analysed by Western blot using an antibody detecting phosphorylation at a specific, PP2A sensitive tau site. Band intensities were normalized to actin and compared to total tau (Tau 5). n=5, * p<0.05.

FIG. 5 shows that Metformin increases PP2A-activity in-vitro. Three independent experiments were measured in A, B and C. (A) Purified PP2Ac was incubated with or without Metformin (10 mM) or okadaic acid (10 nM). PP2A activity was determined in a malachite green assay. (B) immunoprecipitated PP2A-holoenzymes were incubated with or without metformin (10 mM) or okadaic acid (10 nM). PP2A activity was determined in a malachite green assay. (C) Cells were treated with or without metformin (2.5 mM) and lysed. PP2A activity was determined in a malachite green assay. n=3, * p<0.05. (D) Assembly of the PP2Ac-MIDI-α4 protein complex is influenced by Metformin. Affinity chromatography analysis shows the binding of immobilized PP2Ac to the MID1 B-box domains and α4. The Flow through, the first washing fraction, several fractions obtained with a buffer containing different concentrations of metformin and the elution fraction were collected and analyzed on SDS-gels. Gels were stained with Coomassie. (E) Association between PP2Ac and α4 is influenced by metformin. Co-immunoprecipitations of PP2Ac and V5-tagged α4 in the presence or absence of metformin were performed and analyzed on Westernblots using PP2Ac- or V5-specific antibodies. This experiment has been repeated three times.

FIG. 6 shows that Metformin decreases tau phosphorylation in-vivo. Pairs of wildtype mice were fed with or without 5 mg/ml of metformin in the drinking water for 16-24 days. Representative Western blots and quantification of three technical replicates are shown in the subsequent experiments. (A) Brains were lysed and analysed via Western blots for phosphorylation of S6, Band intensities were compared to actin. (B) Brains were lysed and analysed via Western blots for phosphorylation of AMPK (pThr 172), Band intensities were compared to AMPK total. (C) Brains were lysed and analysed via Western blots for phosphorylation of Tau. pSer202 was detected using a phospho-specific pSer202 antibody and a taul antibody, which detects dephosphorylated Ser202. Phosphorylation at position Ser 262 was detected using a phospho-specific Ser262 antibody. Band intensities were normalized to actin and compared to total tau (Tau5). (D) Brains were lysed and analysed via Western blots for phosphorylation of the PP2A insensitive site pSer396. Band intensities were normalized to actin and compared to total tau (Tau5). * p<0.05.

FIG. 7 shows that several phosphorylation sites of the tau protein are PP2A sensitive. Primary cortical neurons of wildtype mice were treated with the PP2A inhibitor okadaic acid (OA) in increasing concentrations. Cell lysates were analysed by Western blot using antibodies detecting phosphorylation at specific tau sites (pSer356, pSer396, pSer262, pSer202). Band intensities were normalized to actin and compared to total tau (Tau 5). Representative Western blots and quantifications of three independent experiments are shown (n=3), n.s. p>0.05.

FIG. 8 shows that Metformin treatment leads to a decrease of pS6kinase phosphorylation. Primary cortical neurons of wildtype mice were treated with 2.5 mM metformin (Met) over increasing time intervals. Cell lysates were analysed by Western blot using an anti-phospho S6kinase (Pp70S6K) antibodies. Band intensities were normalized to actin and compared to total S6kinase protein. Representative Western blots and quantifications of three independent experiments (n=3) are shown, * p≦0.0004.

FIG. 9 shows that Phenformin treatment leads to a decrease of pS6kinase phosphorylation. Primary cortical neurons of wildtype mice were treated with 0.25 mM Phenformin over increasing time intervals. Cell lysates were analysed by Western blot using an anti-phospho S6kinase (Pp70S6K) antibody. Band intensities were compared to total S6kinase protein (p70S6K).

FIG. 10 shows that (A) Phenformin induces de-phosphorylation of tau at PP2A sensitive sites. Primary cortical neurons of wildtype mice were treated with increasing concentration of Phenformin over 16 h or 1 h respectively. Cell lysates were analysed by Western blot using antibodies detecting phosphorylation at specific, PP2A sensitive tau sites. Band intensities were compared to total tau (Tau 5) and actin. (B) Primary cortical neurons of human tau expressing mice were treated with 10 μM of rapamycin over increasing time intervals. Cell lysates were analysed by Western blot using antibodies detecting phosphorylation at a specific, PP2A sensitive tau site. Band intensities were compared to total tau (Tau 5) and actin.

FIG. 11 shows that inhibition of PP2A blocks the Phenformin effect on the phosphorylation of tau. Primary cortical neurons of wildtype mice were treated with either okadaic acid (OA) (10 nM), only Phenformin (Ph) (0.25 mM) or with okadaic acid (10 nM) prior to Phenformin (0.25 mM) over 4 hours. Cell lysates were analysed by Western blot using antibodies detecting phosphorylated (pSer202) or de-phosphorylated tau (Tau-1) at a specific, PP2A sensitive tau site. Bands were normalized to actin.

FIG. 12 shows that Phenformin effects on the phosphorylation of tau are AMPK independent. Primary cortical neurons of wildtype mice were treated with 0.25 mM of Phenformin over increasing time intervals. Cell lysates were analysed by Western blot using an antibody detecting phosphorylation of the AMPK target ACC. Band intensities were compared to total ACC.

FIG. 13 shows that Metformin decreases tau phosphorylation in-vivo. Pairs of wildtype mice were fed with or without 5 mg/ml of metformin in the drinking water for 16-24 days. (A) Brains were lysed and analysed via Western blots for phosphorylation of S6, Band intensities were compared to actin. Representative Western blots and quantification of three technical replicates are shown. *p<0.04 (B) Brains were lysed and analysed via Western blots for phosphorylation of Tau. pSer202 was detected using a phospho-specific pSer202 antibody and a taul antibody, which detects dephosphorylated Ser202. Band intensities were normalized to actin and compared to total tau (Tau5), Representative Western blots and quantification of three technical replicates are shown * p<0.05.

FIG. 14 shows that Metformin increases the activity of GSK3β. Primary cortical neurons of wildtype mice were treated with 2.5 mM metformin (Met) over increasing time intervals or with phenformin over 4 hours with increasing concentrations. Cell lysates were analysed by Western blot using an anti-phospho GSK3β antibody (pSer9). This de-phosphorylation at this site activates the enmzym. Band intensities were compared to total GSK3β and actin.

### DETAILED DESCRIPTION OF THE INVENTION

The present invention now will be described more fully hereinafter in which embodiments of the invention are provided with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art.

The terminology used in the description of the invention herein is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise.

Unless otherwise defined, all technical and scientific terms used herein have the same meaning as commonly understood by one of ordinary skill in the art to which this invention belongs.

### Materials and Methods

### Primary Cultures

Primary cortical neurons were isolated from brain of wt or transgenic (Mapt^{tm1(EGFP)klt} Tg(MAPT)8cPdav/J; Jackson Laboratory) embryos at day 14.5. Cortices were collected in DMEM (Lonza) and cells were dissociated by incubation with Trypsin/EDTA for 6.5 min at 37° C. Cells were then diluted in neurobasal medium (Gibco-BRL) containing B27 supplement (Gibco BRL). Cells were plated at a density of 4.0×10⁵ onto 0.2 mg/ml poly-D-lysin (Sigma) and 2 μg/ml Laminin (Sigma) coated 12 well plates. Neurons were incubated at 37° C. with 8% CO₂. One hour after seeding a complete change of medium was performed. Later on, each 96 hours half volumes of the medium were replaced by fresh medium. After 7 days in culture neurons were treated.

### Treatments

Cells were incubated with the respective substances as follows: Metformin (Sigma) at final concentrations up to 2.5 mM for 1 to >24 hours, okadaic acid (Sigma) at final concentrations from 1 nM to 10 nM for 4 h, Fostricien (Sigma) at final concentration of 1 μM for 4 h and AICAR (cell signalling) at final concentration of 5 mM for 0.5-16 h. For cell treatment without insulin, medium containing B27+insulin supplement was replaced with medium containing B27-supplement without insulin 24 h before treatment. 3 groups of 2 mice were treated with or without 5 mg/l metformin in the drinking water.

### Tissue Preparation

Mice were decapitated and brains were rapidly dissected in nitrogen. Brains were homogenized in ice-cold lysis buffer containing 10 mmol/l Tris pH 7.5, 150 mM NaCl, 1 mM EDTA, 1 mM EGTA, 1% NP40, protease inhibitors(complete mini, Roche), phosphatase inhibitors (PhosStop, Roche). Lysates were sonicated and centrifuged at 25000 g for 10 min at 4° C.

### Western Blot and Antibodies

Cell pellets were homogenized in Magic-Mix (48% UREA, 15 mM Tris-HCL pH 7.5, 8.7% Glycerin, 1% SDS, 0.004% Bromphenolblue, 143 mM Mercaptoethanol), sonicated and boiled for 5 min at 95° C. Proteins were resolved on 8 or 10% SDS gels and blotted onto PVDF-membranes (Roche). Antibodies used in this study were purchased from the following companies: Tau-5 (Biosource), anti-human PHF pSer202 (Thermo scientific), Tau pSer356 (Biosource), Tau pSer262 (Biosource), Tau pSer396 (Sigma), Actin (Sigma), ACC (cell signalling), ACC pSer79 (cell signalling), phospho-S6 ribosomal protein pSer241/244 (cell signalling), p70S6 Kinase (cell signalling), p70S6K pThr421/pSer424 (cell signalling), mTOR (cell signalling), AMPK (cell signallling, AMPK pThr172 (cell singnaling), HRP-anti-rabbit (Amersham), HRP-anti-mouse (Dianova). The resulting bands were quantified using Imagequant5.2. Statistical analyses were performed using SigmaStat software package (v 3.0, SPSS, USA). For timecourse or dose-response studies, data were analyses by one-way ANOVA with post hoc Dunnett's or Bonferonni test to accommodate for multiple comparisons. A Student's t-test was used for two-group comparisons, as appropriate.

### TUNEL Assay

Primary neurons were grown on coverslips with a density of 2×10⁵ per 12 well. TUNEL assays were performed using TUNEL enzyme (Roche) and TUNEL label (Roche) following the manufacturer's instructions.

### PP2A Activity Assay

Either purified PP2Ac (upstate), cell lysates or PP2A-holoenzymes, which were purified by immunoprecipitation using a PP2A-A-subunit antibody (Millipore), were incubated with or without 10 mM metformin or 10 nM okadaic acid for 1 hour. Afterwards PP2A phosphatase activity was determined using a malachite green assay kit (upstate) following the manufacturer's instructions.

### Affinity Chromatography

The MID1-B-box-domains, α4, or HIS-tagged PP2Ac were expressed in E. coli BL21. Cells were lysed by French press in buffer A (50 mM Na-phosphate, 300 mM NaCl, 20 mM imidazole, pH 8.0). Lysates were mixed and PP2A-protein complexes were immobilized on Ni-NTA columns (Qiagen) and washed extensively with buffer A containing 10% glycerol and 0.25% Tween20. After washing columns were eluted with buffer A containing different concentrations of metformin. Elutions were analyzed on SDS-Gels followed by Coomassie staining.

### EXAMPLE 1

### Okadaic Acid Increases Phosphorylation of PP2A Dependent Tau Epitopes

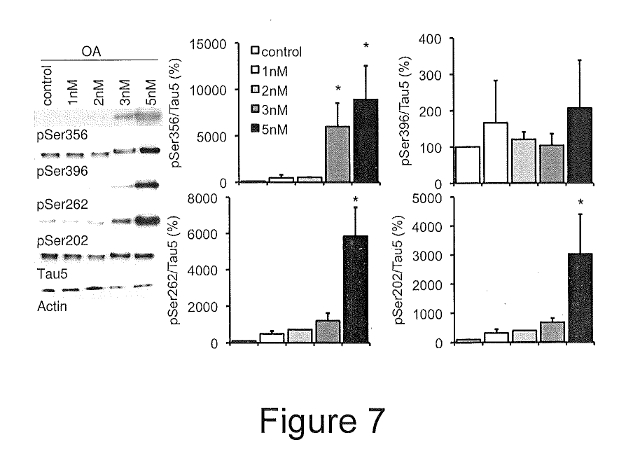

Tau is phosphorylated at numerous serine and threonine residues. Several of these residues have been shown to depend on PP2A activity and be de-phosphorylated by PP2A in-vitro [reviewed in (1, 20, 21)]. In order to confirm these observations in living cells, primary cortical neurons from wild-type mice were treated with increasing concentrations of okadaic acid (OA) for four hours. Protein lysates of treated cells were analysed by Western blot. Phosphorylation of Serine (Ser) 202, Ser262, Ser356, and Ser396 was analysed relative to total tau using specific anti-phospho antibodies and the anti-tau5 antibody detecting total tau. As expected, phosphorylation of Ser202, Ser262, and Ser356 increased with the concentration of OA (FIG. 7) confirming that the phosphorylation of these epitopes is regulated by PP2A or a closely related phosphatase. Phosphorylation of Ser396, however, did not increase significantly in cells treated with OA, which confirms previous observations that Ser396 is not an efficient PP2A target (21).

### EXAMPLE 2

### Metformin Inhibits mTORC1/Activates PP2A

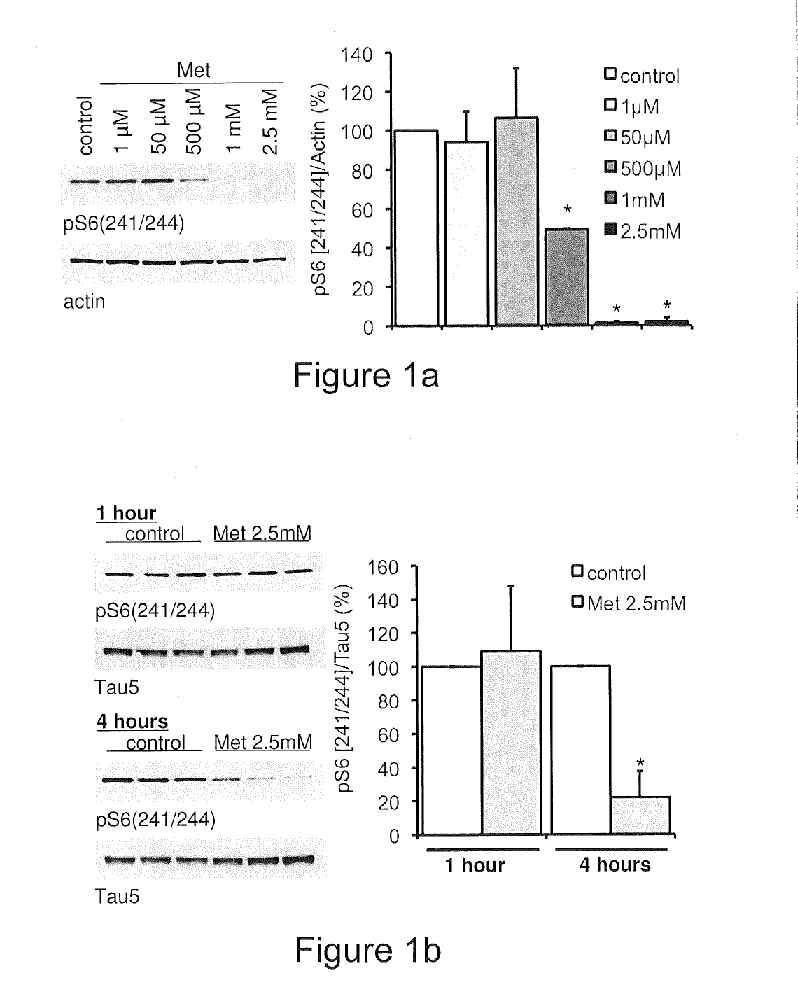

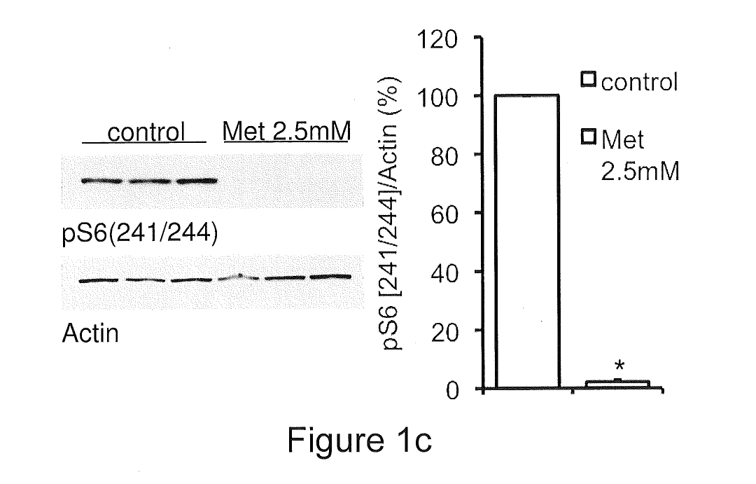

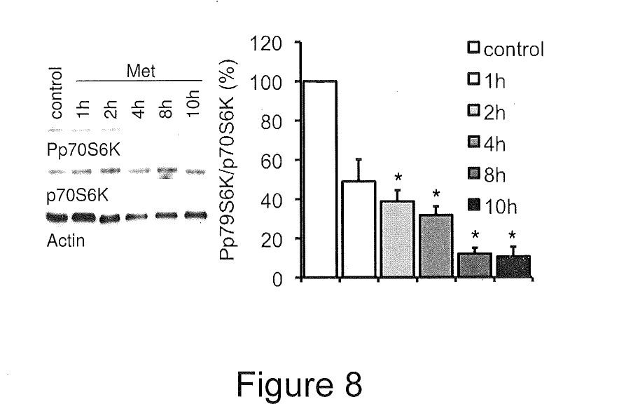

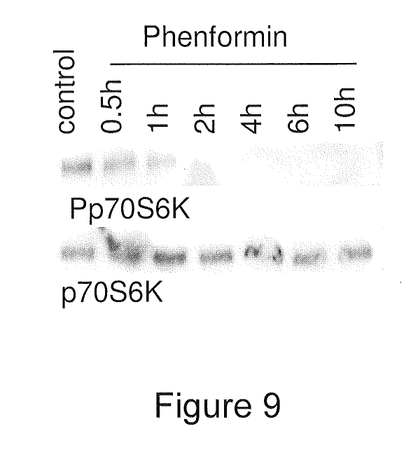

The anti-type II diabetes drug metformin has been shown to inhibit mTOR activity (22, 23). Because PP2A is regulated by mTOR (24), we speculated that metformin would be able to (i) increase PP2A activity and (ii) thereby reduce tau phosphorylation at PP2A dependent epitopes. To confirm that metformin could inhibit mTORC1 signalling in primary neurons from wild-type mice, we measured the phosphorylation of S241/244 of ribosomal protein S6 following incubation with increasing concentrations of metformin (FIG. 1). A dose dependent decrease in S6 phosphorylation was detected (FIG. 1a), and significant dephosphorylation was present within 4 h of exposure to 2.5 mM metformin (FIG. 1b). This ability of metformin to dephosphorylate S6 was not specific to neurons, but clearly apparent in the rat hepatoma cell line H4IIE, which were incubated with metformin for 16 hours (FIG. 1c). Results were confirmed by measuring the phosphorylation status of p70S6kinase, another target of mTORC1/PP2A after metformin treatment (FIG. 8) in primary neurons. Furthermore, phenformin, a metformin derivative, also promoted de-phosphorylation of p70S6kinase with time FIG. 9).

### EXAMPLE 3

### Metformin Induces Dephosphorylation of Tau in Cortical Neurons

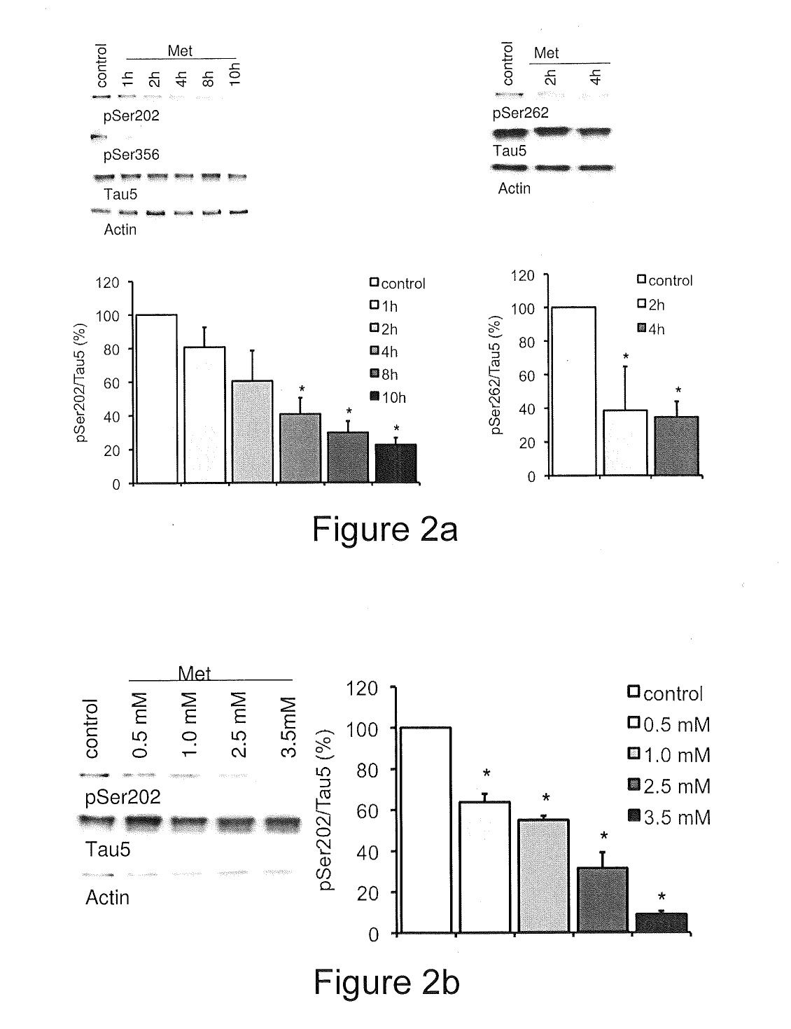

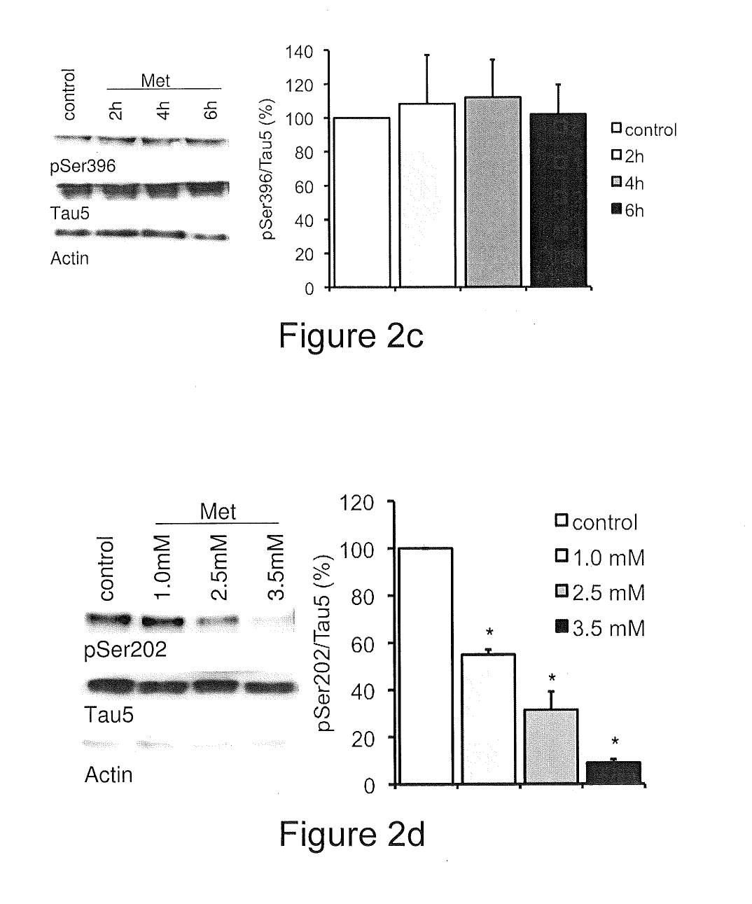

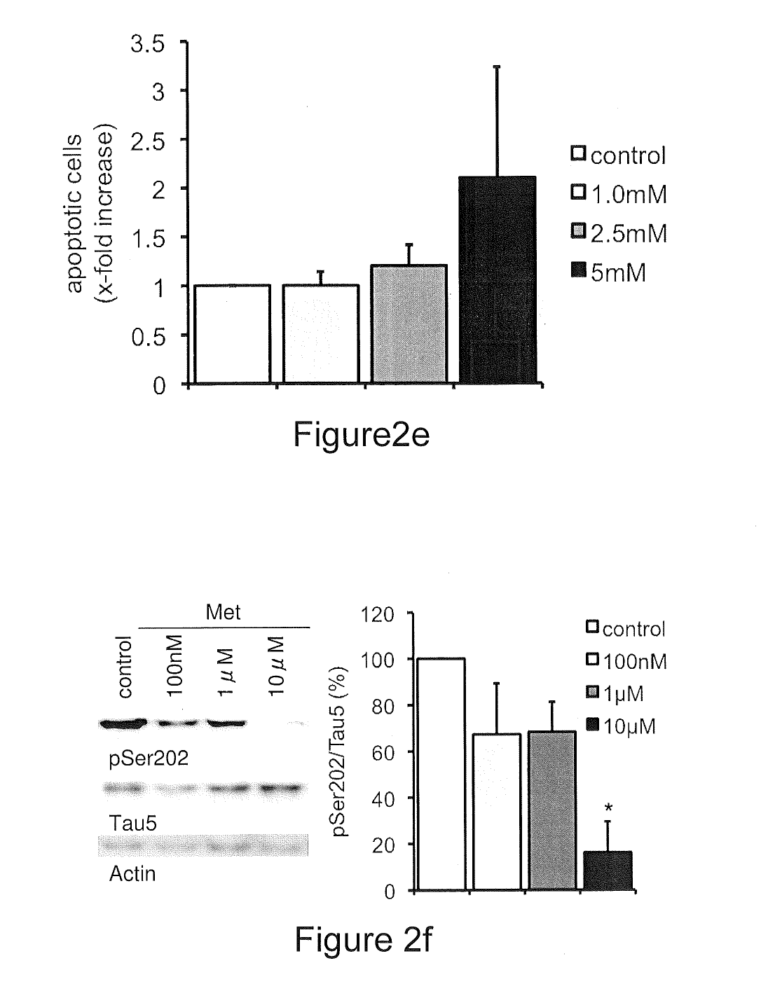

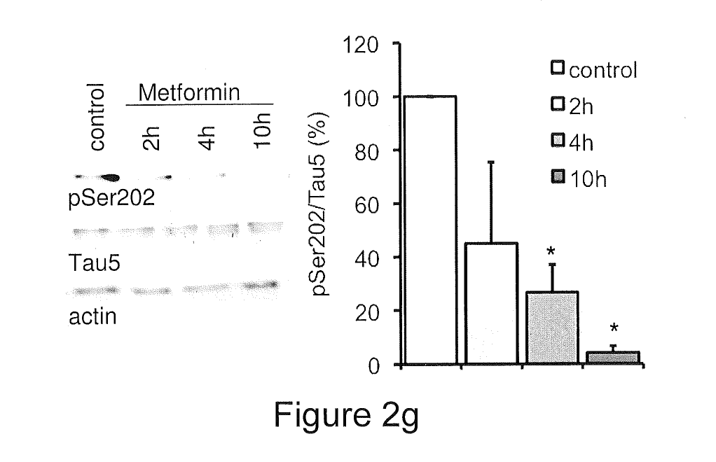

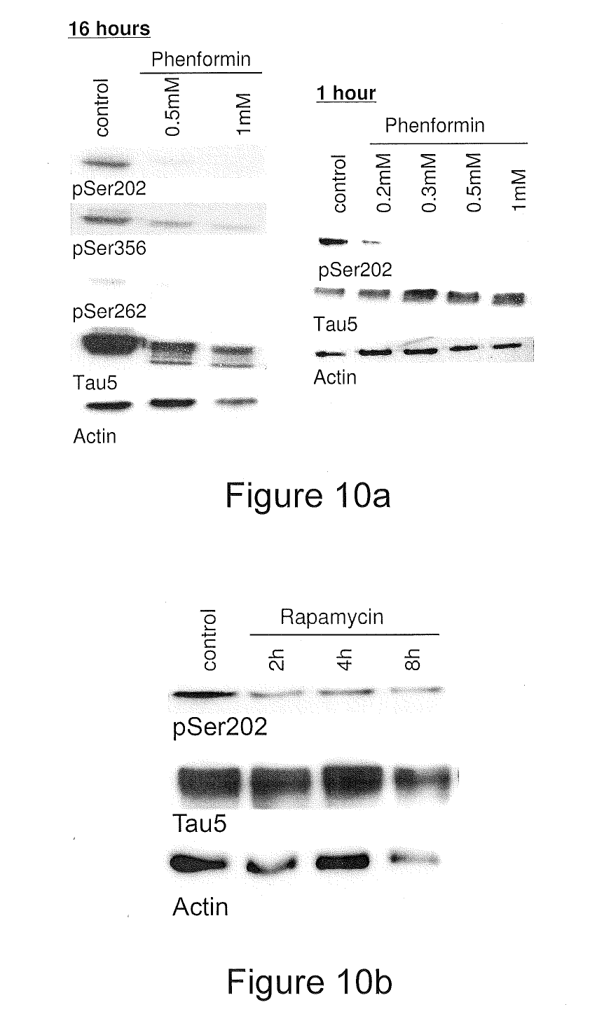

Because activation of PP2A should result in a de-phosphorylation of PP2A dependent tau epitopes, primary cortical neurons from wild-type mice were treated with 2.5 mM metformin for different time intervals and the phosphorylation of the PP2A dependent tau epitopes Ser202, Ser356 and Ser262 was analysed by Western blot. A significant decrease of the phosphorylation of all three epitopes was observed after 2-4 hours. Furthermore, de-phosphorylation of these epitopes increased with time (FIG. 2a) and dosage (FIG. 2b). By contrast, phosphorylation of the PP2A insensitive epitope Ser396 did not change after metformin treatment (FIG. 2c), indicating that metformin only affects PP2A dependent sites. Similar analyses of primary cortical neurons from mice that express the human tau protein instead of the murine showed that human tau epitopes reacted in the same way to metformin treatment (FIG. 2d). Comparable results were obtained when treating cells with the metformin derivative phenformin (FIG. 10a) and with the mTOR inhibitor/PP2A activator rapamycin (FIG. 10b), which adds further proof that indeed, the enzyme couple mTOR/PP2A influences phosphorylation of tau at the sites analysed. In order to make sure that the observed effects are not due to an increase in cellular apoptosis, TUNEL assays with increasing amounts of metformin on primary cortical neurons were performed showing that metformin did not cause a significant increase of programmed cell death in these cells at concentrations sufficient to induce tau dephosphorylation (FIG. 2e).

Metformin has been in clinical use for patients with Type II Diabetes for many years. Peak plasma concentrations at dosages that are being applied to patients, however, are around 12 μM (25). These are much lower than what we have used in the above experiments. Because tau de-phosphorylation increases with time after metformin treatment, in the next series of experiments metformin concentrations were reduced significantly using longer treatment periods (>24 hours) instead. Again, primary cortical neurons of wild-type mice were treated with the respective amounts of metformin and subsequently analysed for phosphorylation at the PP2A dependent tau site Ser202. Already at 100 nM metformin, phosphorylation of Ser202 decreased slightly, while at 10 μM phosphorylation of Ser202 is significantly reduced (FIG. 2f).

Metformin effects have been shown to be insulin sensitive. In order to rule out an insulin-dependent effect on tau phosphorylation we incubated primary neurons with 2.5 mM metformin this time using medium without insulin. As in the previous experiments metformin had a strong dephosphorylation effect on tau (FIG. 2g) suggesting that metformin acts in an insulin independent way.

### Metformin Functions Through PP2A Activity

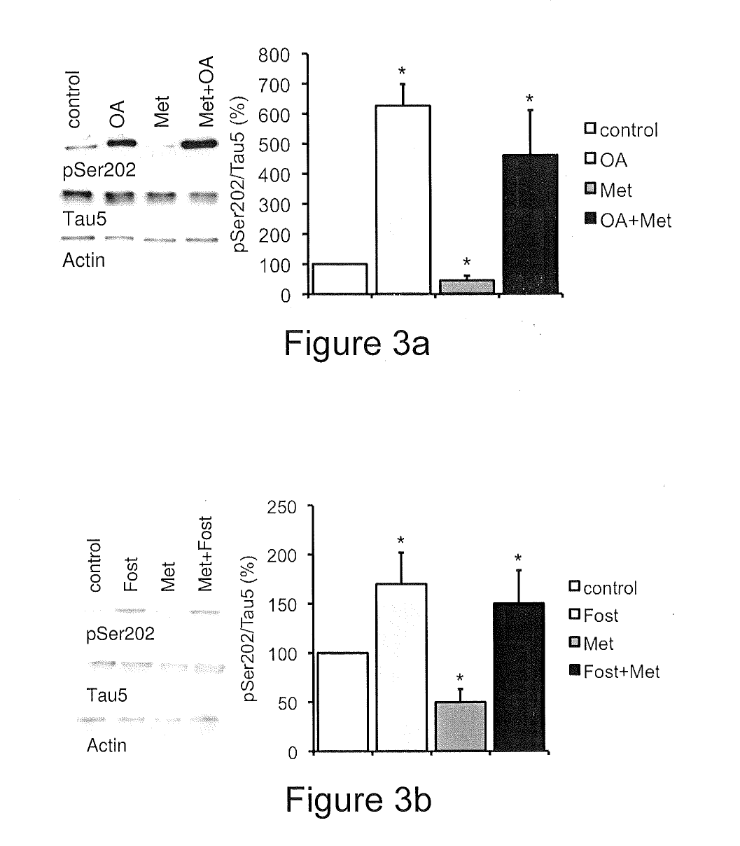

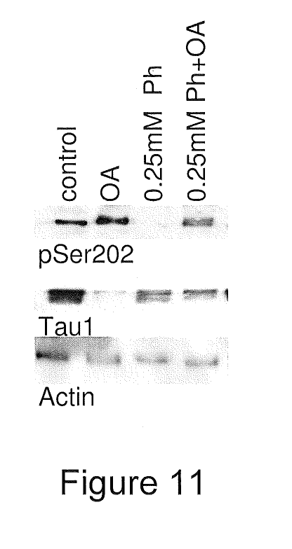

In order to show that metformin requires PP2A activity to regulate tau dephosphorylation, primary cortical neurons of wild-type mice were incubated with or without 10 nM okadaic acid prior to metformin treatment. In confirmation of our previous results, phosphorylation of Ser202 increased after okadaic acid treatment and decreased significantly after treatment with 2.5 mM metformin over 4 hours. By contrast, metformin had no effect on the Ser202 phosphorylation pattern of okadaic acid treated cells (FIG. 3a) indicating that the metformin effect on the phosphorylation of tau depends on PP2A activity. Similar data were generated with the metformin derivative phenformin (FIG. 11). In confirmation of the okadaic acid data primary neurons were also treated with the more specific PP2A inhibitor fostriecin. Again, while phosphorylation at Ser202 increased after treatment with 1 μM fostriecin over 4 hours and decreased after treatment with 2.5 mM metformin over the same period of time, metformin did not have any effect on fostriecin pre-treated cells (FIG. 3b).

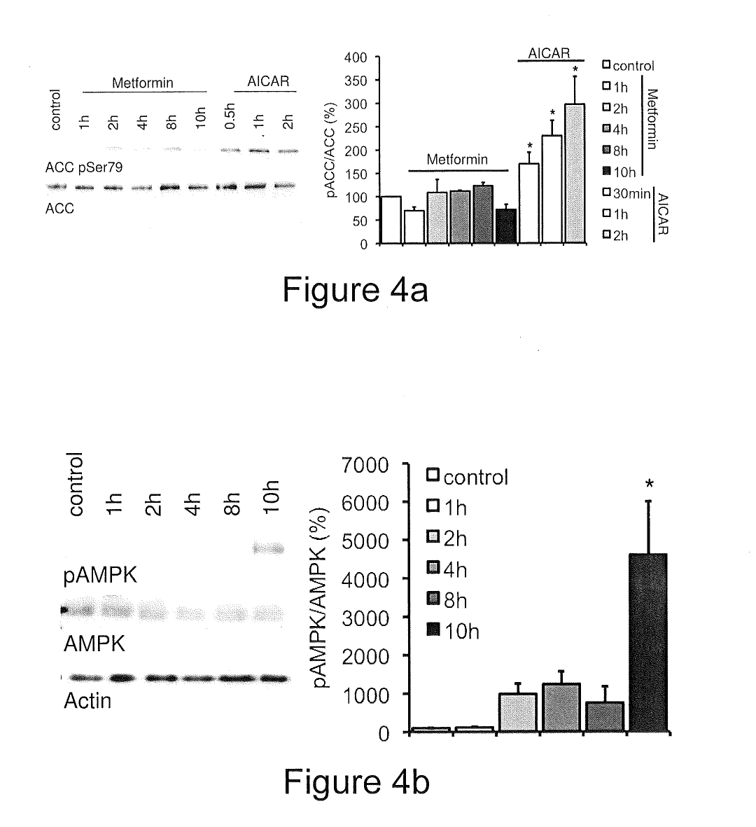

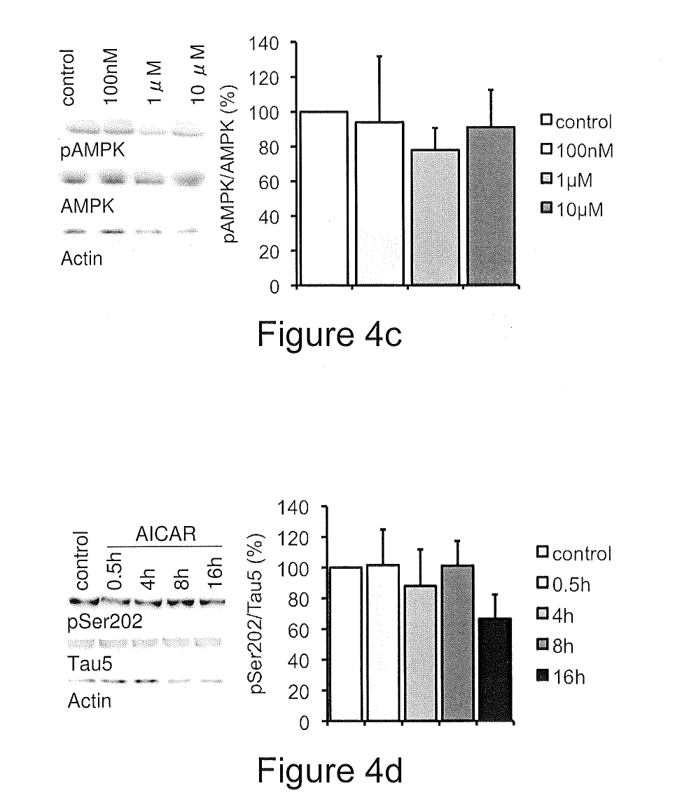

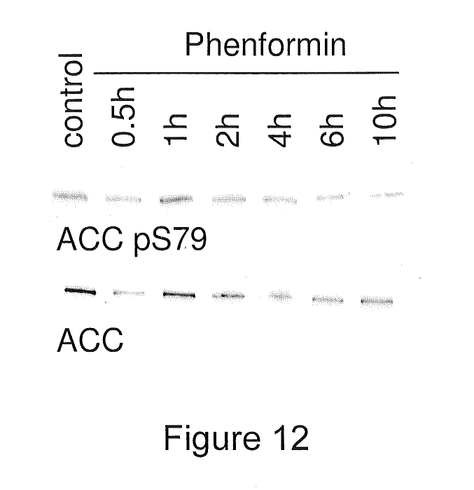

Because metformin had been shown in other systems to activate AMPK, an antagonist of mTOR, we assumed that metformin would activate PP2A via AMPK activation. To examine this hypothesis, primary cortical neurons were treated with 2.5 mM of metformin and analysed by Western blot for the phosphorylation of the AMPK target ACC using an antibody detecting phosphorylated Ser79. The AMPK agonist AICAR was used as a positive control. To our surprise, while ACC phosphorylation significantly increased after AICAR treatment, neither metformin (FIG. 4a) nor phenformin (FIG. 12) had an effect on the phosphorylation pattern of ACC. Analysis of the phosphorylation pattern of AMPK itself showed that metformin does induce AMPK phosphorylation significantly after incubation with 2.5 mM metformin for 10 hours (FIG. 4b). However, no significant effect was seen after shorter intervals of incubation with 2.5 mM metformin or when using smaller amounts of metformin for a time period of 24 hours (FIG. 4c) indicating that metformin does not induce AMPK activity in primary cortical neurons in the concentration and the time intervals that are necessary for PP2A activation and tau dephosphorylation. Furthermore, we did not find a significant effect of the AMPK agonist AICAR on the phosphorylation pattern of tau during the first 16 hours of treatment (FIG. 4d), which further indicates that AMPK activation alone is not sufficient to mimic the effect of metformin on tau and that metformin could mediate tau de-phosphorylation independently of AMPK.

### EXAMPLE 4

### Metformin Interferes with the Composition of the PP2A Protein Complex

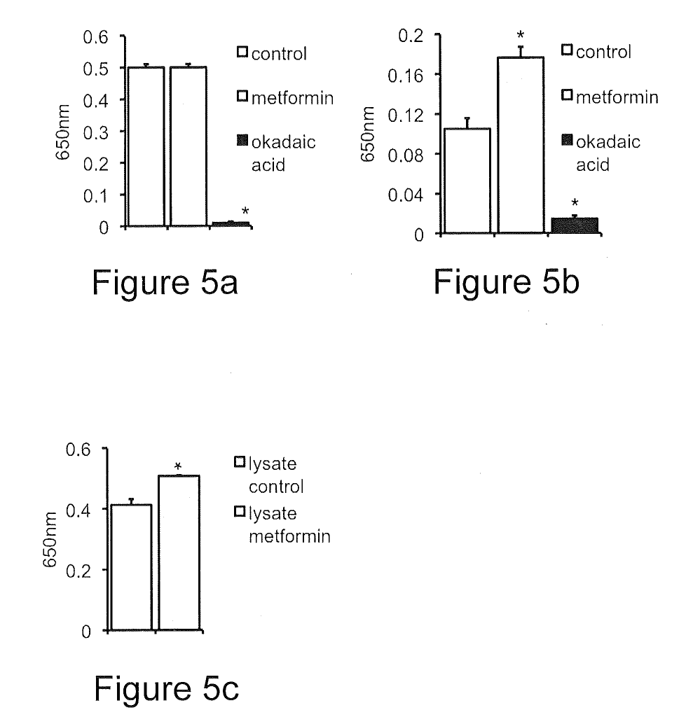

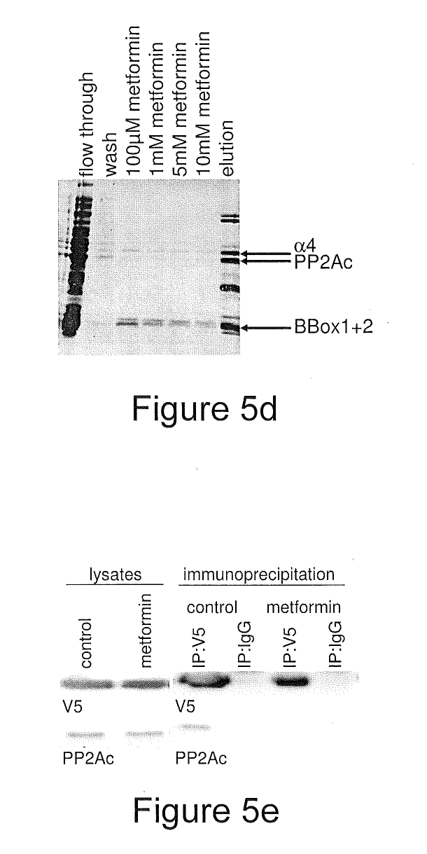

In order to analyse if metformin has direct effects on PP2A activity either purified PP2Ac (catalytic subunit of PP2A) or PP2A-holoenzymes, that were immunopurified from HeLa cell lysates using a PP2A-A subunit specific antibody, were incubated with or without 10 mM metformin or 10 nM okadaic acid. Subsequently PP2A activity was measured with a commercial PP2A activity assay. While no influence of metformin on purified PP2Ac was measured (FIG. 5a), activity of the immunopurified PP2A protein complex was significantly increased after metformin incubation (FIG. 5b) indicating that the metformin effect on PP2A activity is mediated by a component of the PP2A protein complex. The PP2A inhibitor okadaic acid reduced the activity of purified PP2Ac and the PP2A immunocomplex significantly. Furthermore, we have treated primary neurons with metformin and analyzed PP2A activity in the cell lysates. Again, metformin treatment led to a significantly increased phosphatase activity (FIG. 5c).

PP2A activity can be regulated by association of PP2Ac with the .α4 protein. α4 binds to the B-box-domains of the MID1 protein and thereby triggers the binding of the microtubule-associated pool of PP2Ac to the ubiquitin ligase MID1. Via this interaction MID1 can ubiquitinate PP2Ac and induce its proteasomal degradation, thereby regulating the activity of microtubule associated PP2Ac (26). To test, if metformin might induce the activity of PP2A by interfering with its negative regulators MID1 and α4, we analyzed the binding of the protein complex in an in-vitro assay. All three proteins (α4, the B-box-domains of MID1, and PP2Ac) were expressed in E. coli and mixed to allow protein complex assembly. Afterwards the complexes were bound to a column via the HIS-tagged PP2Ac. Interestingly, both, the B-boxes and α4 could be eluted from the column by adding metformin to the washing buffer (FIG. 5d). To confirm that metformin inhibits the assembly of the MID1-α4-PP2Ac complex in cells we performed co-immunoprecipitation experiments using overexpressed V5-tagged α4 protein and analyzed the amount of PP2Ac that is associated with α4 in the presence or absence of 2.5 mM metformin. Clearly, addition of 2.5 mM metformin to lysates of α4-V5 expressing HeLa cells led to a dissociation of α4 from PP2Ac (FIG. 5e). Taken together these data suggest that metformin activates PP2A-activity by inhibiting the binding of PP2Ac to its negative regulators MID1 and α4.

### EXAMPLE 5

### Metformin Induces Dephosphorylation of Tau In-Vivo

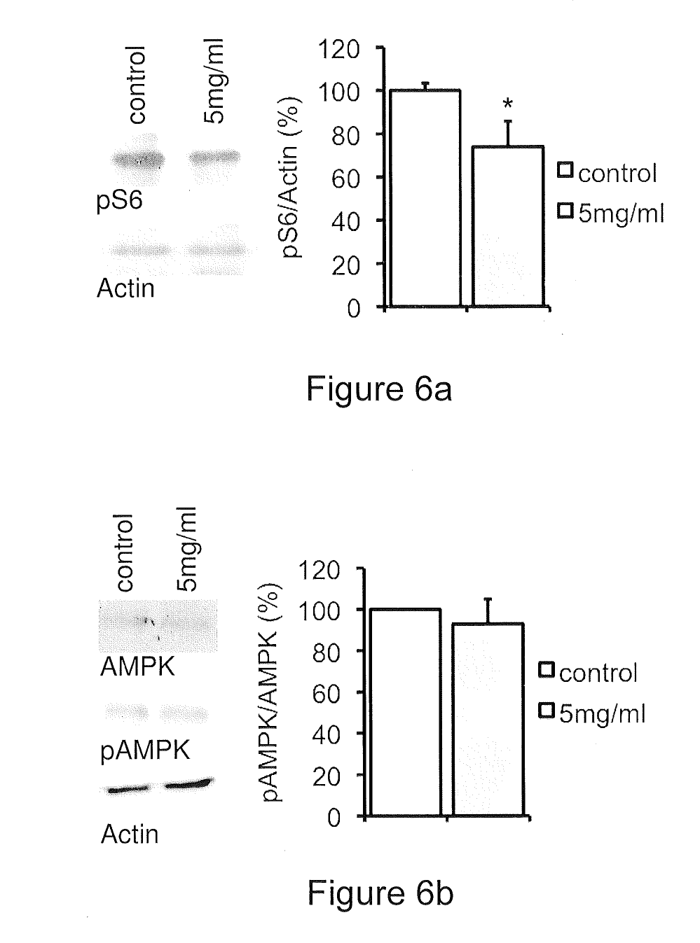

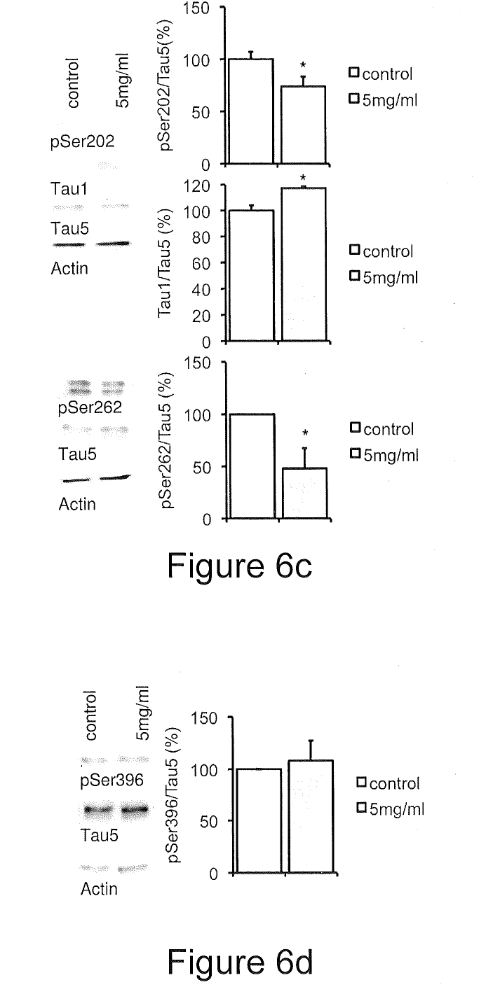

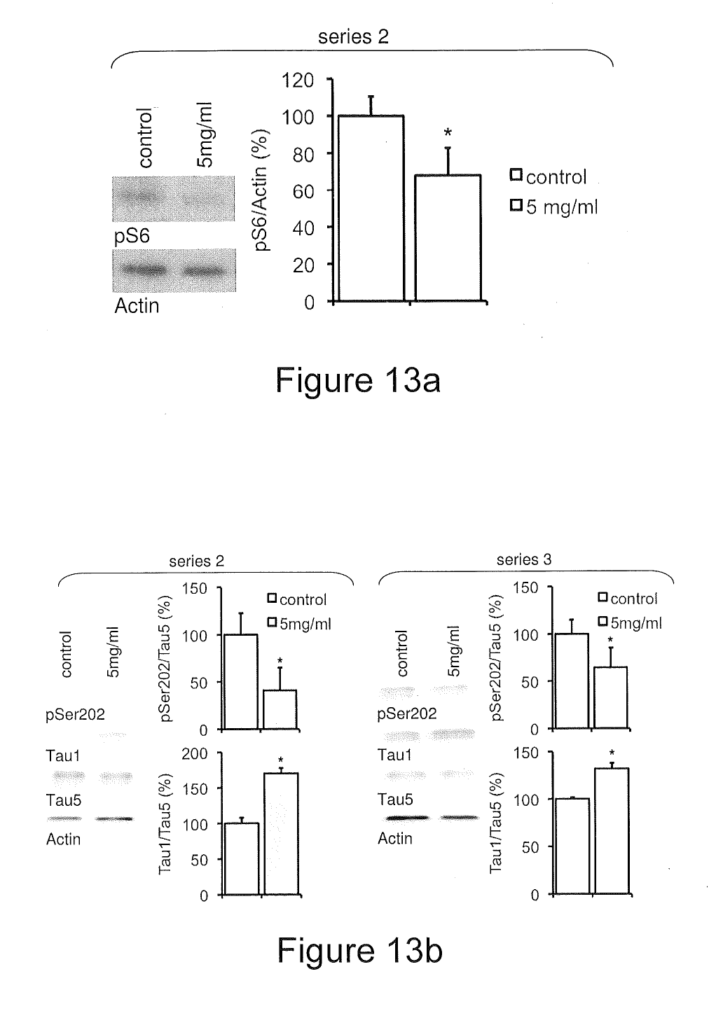

Metformin had previously been shown to be active in the brain after oral administration (27, 28). Chen et al have found that a dosage of only 2 mg/ml in the drinking water fed to mice for 6 days would cause equivalent levels of 1 μM of metformin in the brain. To see if metformin would activate PP2A and de-phosphorylate tau in-vivo, we fed pairs of mice with or without 5 mg/ml of metformin in the drinking water for 16-24 days. Brains were lysed and analysed via Western blot for phosphorylation of S6 and AMPK. While no induction of AMPK phosphorylation was seen, S6 was significantly de-phosphorylated in the metformin treated samples (FIG. 6a, b, FIG. 13a). Phosphorylation of tau was tested at Ser202 using both, an antibody detecting the phosphorylated form and the tau-1 antibody that detects tau dephosphorylated at the same site. In addition epitopes Ser262 and the PP2A independent epitope Ser396 were analysed. A significant decrease of Ser202 and Ser262 phosphorylation and an increase of the de-phosphorylated form of tau at position Ser 202 were observed in the metformin treated mice (FIG. 6c, FIG. 13b) indicating that metformin has an in-vivo effect on the phosphorylation of tau. In confirmation with the data in the primary neurons no effect of metformin on Ser 396 was detected (FIG. 6d).

### Discussion:

Hyperphosphorylation of the microtubule-associated tau protein is a major component in the pathogenesis of AD and related tauopathies. We show here that, in confirmation with the literature, several of the sites that are hyperphosphorylated in AD respond to okadaic acid treatment, an inhibitor of PP2A activity. Furthermore we show that the anti-type II diabetes medication metformin and its derivative phenformin activate PP2A and efficiently de-phosphorylate tau in-vitro and in-vivo in a PP2A dependent manner.

PP2A is regulated by the kinase mTOR (19) and both enzymes affect the phosphorylation status of p70S6kinase and the ribosomal protein S6. As we show here, the phosphorylation status of p70S6kinase and S6 is reduced after metformin treatment. However, it is difficult to distinguish, if metformin activates PP2A or, as had been postulated before in cancer cells, primarily inhibits mTOR activity (22, 29). Here we found that metformin has a direct effect on the association between PP2Ac, the regulatory subunit of PP2A-α4- and the ubiquitin ligase MID1, which targets microtubule associated PP2A for degradation via the proteasome. Our data suggest that metformin interferes with PP2A activity directly, by inhibiting its proteasomal degradation rather than via mTOR inhibition.

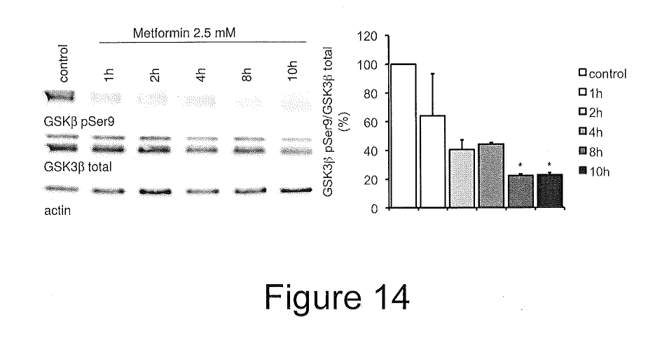

In a recent paper Meske and colleagues have demonstrated that PP2A activity and GSK3β—which is the PP2A counteracting kinase on many tau phosphorylation sites—are tightly regulated in neurons (30). Consistent with their data we did not see an effect of the AMPK agonist AICAR on the phosphorylation of tau (Ser202) in the first 8 hours of treatment, although AMPK is activated within that time. Furthermore we found that metformin reduces the phosphorylation of GSK3β at position Ser 9 thereby activating the enzyme (FIG. 14). Taken together these data and our observations suggest that, (i) in primary neurons, metformin has an acute and prolonged effect on the activity of PP2A, which is likely to overcome GSK3β counter-regulation of tau phosphorylation and (ii) AMPK activation alone is not sufficient to mimic the action of metformin on the phosphorylation of tau.

It is perhaps surprising that we found that metformin and phenformin effects on tau phosphorylation in primary cortical neurons seem to be largely independent of AMPK. It is generally accepted that metformin, by inhibiting complex I of the respiratory chain, leads to a rise of cellular AMP:ATP ratios. AMP binding to AMPK increases AMPK phosphorylation by LKB1, which then results in an increase of AMPK activity [reviewed in (31)]. However, there is growing evidence that metformin also functions in an AMPK independent manner in several cell systems. Thus, metformin has been shown to suppress the over-expressed oncoprotein HER2 in breast cancer cells (22) and to decrease prostate tumour cell growth by influencing cyclin D1 levels (32). In both cases, metformin effects are not abolished by AMPK inhibition. HER2 suppression, however, can sufficiently be blocked by knock-down of the PP2A and mTOR target p70S6kinase, pointing at a PP2A/mTOR dependent mechanism underlying metformin function. Also some of the metabolic effects seen in isolated working rat hearts and cultures of heart-derived cells after metformin treatment clearly seem AMPK independent (33). In accordance with all these data we show here that metformin has an immediate effect on the phosphorylation of the PP2A/mTOR targets p70S6kinase, S6, and of tau, but does not show a significant influence on the phosphorylation of the AMPK target ACC and only a comparably small effect on AMPK itself in primary cortical neurons at the concentrations used here. Its effects are efficiently blocked by the PP2A inhibitors okadaic acid and fostriecin. Furthermore, while metformin (and its derivative phenformin) reduce tau phosphorylation acutely, the AMPK agonist AICAR does not seem to have a comparable effect. This points to an AMPK-independent mode of action of metformin on PP2A activity, or at least to one requiring more than simply AMPK activation.

Rapamycin sensitive association of the catalytic PP2A subunit with the yeast protein TAP42 or its mammalian homologue α4 plays an important role in the regulation of PP2A activity and is probably the most important link between mTOR and PP2A. Importantly, inhibition of mTOR by rapamycin leads to the dissociation of PP2A and α4. The interaction between PP2A and α4 is an unconventional association which only involves the catalytic subunit but not the structural A subunit and seems to influence PP2A activity in a tissue dependent way (34-36). We show here that metformin influences the association between PP2A and α4 thereby possibly inducing PP2A activity.

Recently growing evidence for beneficial effects of metformin on diseases other than diabetes have been presented. Particularly metformin long-term use is associated with lower risk of certain cancers (37, 38). In contrary to such beneficial effects, metformin has been found to induce BACE1 transcription and to increase Aβ production in neuronal cell lines and primary neurons in the absence of insulin (28). Aβ forms the core of amyloid plaques, which are the second pathogenic hallmark in brains of AD patients. However, these effects can be inverted by the addition of insulin leaving the question open of what effects metformin would cause on BACE1 expression and Aβ production in an individual with normal insulin levels. Our data show a significant insulin-independent influence of metformin and its derivative phenformin on the phosphorylation pattern of the AD related tau protein, both after acute and chronic treatment and in-vitro and in-vivo. Our data therefore suggest a potential beneficial effect of long-term metformin treatment and raise the hope that metformin would have a neuro-protective and prophylactic effect in patients with the predisposition for AD.

### REFERENCES

1. Gong C X, Liu F, Grundke-Iqbal I, & Iqbal K (2005) Post-translational modifications of tau protein in Alzheimer's disease. (Translated from eng) J Neural Transm 112(6):813-838 (in eng).
2. Alonso A D, Grundke-Iqbal I, Barra H S, & Iqbal K (1997) Abnormal phosphorylation of tau and the mechanism of Alzheimer neurofibrillary degeneration: sequestration of microtubule-associated proteins 1 and 2 and the disassembly of microtubules by the abnormal tau. (Translated from eng) Proc Natl Acad Sci USA 94(1):298-303 (in eng).
3. Hanger D P, Betts J C, Loviny T L, Blackstock W P, & Anderton B H (1998) New phosphorylation sites identified in hyperphosphorylated tau (paired helical filament-tau) from Alzheimer's disease brain using nanoelectrospray mass spectrometry. (Translated from eng) J Neurochem 71(6):2465-2476 (in eng).
4. Goedert M, Jakes R, Qi Z, Wang J H, & Cohen P (1995) Protein phosphatase 2A is the major enzyme in brain that dephosphorylates tau protein phosphorylated by proline-directed protein kinases or cyclic AMP-dependent protein kinase. (Translated from eng) J Neurochem 65(6):2804-2807 (in eng).
5. Sontag E, Nunbhakdi-Craig V, Lee G, Bloom G S, & Mumby M C (1996) Regulation of the phosphorylation state and microtubule-binding activity of Tau by protein phosphatase 2A. (Translated from eng) Neuron 17(6):1201-1207 (in eng).
6. Gong C X, et al. (2000) Regulation of phosphorylation of neuronal microtubule-associated proteins MAP1b and MAP2 by protein phosphatase-2A and -2B in rat brain. Brain Res 853(2):299-309.
7. Bennecib M, Gong C X, Grundke-Iqbal I, & Iqbal K (2000) Role of protein phosphatase-2A and -1 in the regulation of GSK-3, cdk5 and cdc2 and the phosphorylation of tau in rat forebrain. (Translated from eng) FEBS Lett 485(1):87-93 (in eng).
8. Kins S, et al. (2001) Reduced protein phosphatase 2A activity induces hyperphosphorylation and altered compartmentalization of tau in transgenic mice. (Translated from eng) J Biol Chem 276(41):38193-38200 (in eng).
9. Gong C X, Singh T J, Grundke-Iqbal I, & Iqbal K (1993) Phosphoprotein phosphatase activities in Alzheimer disease brain. (Translated from eng) J Neurochem 61(3):921-927 (in eng).
10. Gong C X, et al. (1995) Phosphatase activity toward abnormally phosphorylated tau: decrease in Alzheimer disease brain. (Translated from eng) J Neurochem 65(2):732-738 (in eng).
11. Vogelsberg-Ragaglia V, Schuck T, Trojanowski J Q, & Lee V M (2001) PP2A mRNA expression is quantitatively decreased in Alzheimer's disease hippocampus. (Translated from eng) Exp Neurol 168(2):402-412 (in eng).
12. Loring J F, Wen X, Lee J M, Seilhamer J, & Somogyi R (2001) A gene expression profile of Alzheimer's disease. (Translated from eng) DNA Cell Biol 20(11):683-695 (in eng).
13. Sontag E, et al. (2004) Altered expression levels of the protein phosphatase 2A ABalphaC enzyme are associated with Alzheimer disease pathology. J Neuropathol Exp Neurol 63(4):287-301.
20. Trojanowski J Q & Lee V M (1995) Phosphorylation of paired helical filament tau in Alzheimer's disease neurofibrillary lesions: focusing on phosphatases. Faseb J 9(15):1570-1576.
21. Wang J Z, Grundke-Iqbal I, & Iqbal K (2007) Kinases and phosphatases and tau sites involved in Alzheimer neurofibrillary degeneration. (Translated from eng) Eur J Neurosci 25(1):59-68 (in eng).
22. Vazquez-Martin A, Oliveras-Ferraros C, & Menendez J A (2009) The antidiabetic drug metformin suppresses HER2 (erbB-2) oncoprotein overexpression via inhibition of the mTOR effector p70S6K1 in human breast carcinoma cells. (Translated from eng) Cell Cycle 8(1):88-96 (in eng).
23. Dowling R J, Zakikhani M, Fantus I G, Pollak M, & Sonenberg N (2007) Metformin inhibits mammalian target of rapamycin-dependent translation initiation in breast cancer cells. (Translated from eng) Cancer Res 67(22):10804-10812 (in eng).
24. Goldberg Y (1999) Protein phosphatase 2A: Who shall regulate the regulator? Biochemical Pharmacology 57(4):321.
25. Goodman & Gilman's (The Pharmacological Basis of Therapeutics.
26. Trockenbacher A, et al. (2001) MID1, mutated in Opitz syndrome, encodes an ubiquitin ligase that targets phosphatase 2A for degradation. (Translated from eng) Nat Genet 29(3):287-294 (in eng).
27. Ma T C, et al. (2007) Metformin therapy in a transgenic mouse model of Huntington's disease. Neurosci Lett 411(2):98-103.
28. Chen Y, et al. (2009) Antidiabetic drug metformin (GlucophageR) increases biogenesis of Alzheimer's amyloid peptides via up-regulating BACE1 transcription. (Translated from eng) Proc Natl Acad Sci USA 106(10):3907-3912 (in eng).
29. Erdemoglu E, Guney M, Giray S G, Take G, & Mungan T (2009) Effects of metformin on mammalian target of rapamycin in a mouse model of endometrial hyperplasia. (Translated from eng) Eur J Obstet Gynecol Reprod Biol 145(2):195-199 (in eng).
30. Meske V, Albert F, & Ohm T G (2008) Coupling of mammalian target of rapamycin with phosphoinositide 3-kinase signaling pathway regulates protein phosphatase 2A- and glycogen synthase kinase-3-dependent phosphorylation of Tau. (Translated from eng) J Biol Chem 283(1):100-109 (in eng).
31. Hardie D G (2003) Minireview: the AMP-activated protein kinase cascade: the key sensor of cellular energy status. (Translated from eng) Endocrinology 144(12):5179-5183 (in eng).
32. Ben Sahra I, et al. (2008) The antidiabetic drug metformin exerts an antitumoral effect in vitro and in vivo through a decrease of cyclin D1 level. (Translated from eng) Oncogene 27(25):3576-3586 (in eng).
33. Saeedi R, et al. (2008) Metabolic actions of metformin in the heart can occur by AMPK-independent mechanisms. (Translated from eng) Am J Physiol Heart Circ Physiol 294(6):H2497-2506 (in eng).
34. Inui S, et al. (1998) Ig receptor binding protein 1 (alpha4) is associated with a rapamycin-sensitive signal transduction in lymphocytes through direct binding to the catalytic subunit of protein phosphatase 2A. Blood 92(2):539-546.
35. Nanahoshi M, et al. (1998) Regulation of Protein Phosphatase 2A Catalytic Activity by alpha4 Protein and Its Yeast Homolog Tap42. Biochemical and Biophysical Research Communications 251(2):520.
36. Kong M, Ditsworth D, Lindsten T, & Thompson C B (2009) Alpha4 is an essential regulator of PP2A phosphatase activity. (Translated from eng) Mol Cell 36(1):51-60 (in eng).
37. Evans J M, Donnelly L A, Emslie-Smith A M, Alessi D R, & Morris A D (2005) Metformin and reduced risk of cancer in diabetic patients. Bmj 330(7503):1304-1305.
38. Currie C J, Poole C D, & Gale E A (2009) The influence of glucose-lowering therapies on cancer risk in type 2 diabetes. (Translated from eng) Diabetologia 52(9):1766-1777 (in eng).

1. A method of preventing and/or treating a tau related disorder in a patient comprising the step of administering to a subject a compound which is capable of inhibiting assembly of a MIDI-α4-PP2Ac complex.

2. The method according to claim 1, comprising administering one or more compounds or classes selected from the group consisting of Biguanides, Guanidines, Berberine, Alkylphosphocholin (Erucylphosphohomocholine), Spermidine, Catechines, Quercetin, Naringin, Apigenin, Luteolin, Procyanidin, Isoflavones: including Genistein, and Daizein, Resveratrol, (Dihydroxyphenyl)ethanol, Curcumin, Alpha-Tocopherol, Forskolin, Inositol, Li+, Troglitazone, Valproate, Ceramide, Alkylphospholipide, Palmitate, Linoleic acid, Phosphatidic acid, Lysophosphatidic acid , FTY720, Endostatin, Vasoinhibin, Somatostatin, Adiponectin, SAM, methionine, THF, Natriumselenate, Sulforaphane, Salicylic acid and Apomorphine.

3. The method according to claim 1 comprising administering one or more biguanidine compound(s) or derivative(s), selected from the group consisting of Metformin, Phenformin, Buformin and Proguanil.

4. The method according to claim 3, the one or more biguanidine compound(s) or derivative(s) is Metformin and/or Phenformin.

5. The method according to claim 1, wherein the tau-related disorder is selected from the group consisting of Progressive supranuclear palsy, Dementia pugilistica (chronic traumatic encephalopathy), Frontotemporal dementia, Parkinsonism linked to chromosome 17 without detectable β-amyloid plaques, Lytico-Bodig disease (Parkinson-dementia complex of Guam), Tangle-predominant dementia, Ganglioglioma and gangliocytoma, Meningioangiomatosis, Subacute sclerosing panencephalitis, Lead encephalopathy, Tuberous sclerosis, Hallervorden-Spatz disease, and Lipofuscinosis

6. The method according to claim 1, wherein the tau-related disorder is Alzheimer's disease.

7. The method according to claim 1, wherein the compound which is capable of inhibiting assembly of a MID1-α4-PP2Ac complex is administered in combination with an additional therapeutic agent or agents.

8. The method according to claim 6, wherein the additional therapeutic agent is an agent designed to target amyloid plaques.

9. The method of claim 1, wherein the patient is a human.
